(19) United States
(12) Patent Application Publication
(10) Pub. No.: US 2006/0198618 A1
(43) Pub. Date: Sep. 7, 2006

(54) Replacement refill kit for a waterless vaporizing device

(76) Inventor: Alan I. Freidel, Miami, FL (US)

Correspondence Address: CARTER, DELUCA, FARRELL & SCHMINDT, LLP, Suite 225, 445 Broad Hollow Road, Melville, NY, 11747, US

(21) Appl. No.: 11/073,128

(22) Filed: Mar. 4, 2005

(51) Int. Cl. A61M 16/00

(52) U.S. Cl. 392/390

(57) ABSTRACT

A kit for replacing a refill pad for a waterless vaporizing unit that emits a vapor has a number of applicator pads and a scented liquid. The scented liquid is for deposit on each applicator pad. The kit has both pads and liquid to be used to fill the pads for a universal replacement of the pad for any number of different waterless vaporizing units.

### BACKGROUND OF THE INVENTION

1. Field of the Invention

The present invention relates to a kit for a waterless vaporizing device. More particularly, the present invention is directed to a kit having one or a number of sized or sizable pads for universal replacement of a used refill pad. Even more particularly, the present invention is directed to a kit for selectively changing one or more scents of a waterless vaporizing or humidifier device.

2. Description of the Related Art

Commercial waterless vaporizing devices are known in the art. One such waterless vaporizing device that is common to the present inventor is U.S. Pat. No.:5,970,212 and U.S. Pat. No.:6,374,044 to Freidel which are both herein incorporated by reference in their entirety. The '212 patent provides for a waterless vaporizing device that has a casing with a heater that causes vapor to be emitted through and from a refill pad. The waterless vaporizing device further has a chimney member that has a pad retainer or holder and a compartment. The refill pad that is saturated with liquid is selectively retained in or on the pad holder in the compartment.

After a period of time the refill pad that is saturated with the oil or scented material becomes less saturated and loses the scent and needs replacement. Alternatively, after a period of time, the user of the device may become tired of the scented vapor and desire a new scent emitted from the device. Still further, the user may have multiple or different vaporizing devices and a desire to use refill pads from another or second sized device in the compartment of a first device. Typically, each waterless vaporizing device manufactures a predetermined sized pad to fit only in the compartment of the particular waterless vaporizing device. This forces the user of the waterless vaporizing device to have to purchase the particular sized pad for the particular waterless vaporizing device and specifically not other pads. This is very detrimental to the user because certain establishments only carry certain brands and the user will have to shop at certain establishments that may cause inconvenience for the user. Still further, the user will be limited to the scents associated with those sold with that particular waterless vaporizing device and will be precluded from other desirable scents. Still further, the user will be limited to paying increased costs for unusual sized pads because the user has no choice but to purchase these pads in conformance with the manufacturer's sized compartment and will be precluded from purchasing other cheaper or more desirable pads as they will not fit in the compartment.

Accordingly, there is a need in the art for a kit for a universal replacement of refill pads and for modulating the scent of existing refill pads in the art. There is also a need for such a kit that eliminates one or more of the aforementioned drawbacks and deficiencies of the prior art and provide convenience to the user.

### SUMMARY OF THE INVENTION

It is an object of the present invention to provide a kit for a waterless vaporizing device that can easily modulate or change a scent for the vaporizing device from one scent to another.

It is another object of the present invention to provide a kit for a waterless vaporizing device that can easily and universally replace any used or spent refill pad from the waterless vaporizing device.

It is yet another object of the present invention to provide a kit for a waterless vaporizing device that has a refill pad or a number of refill pads that can fit or be sized to fit any number of differently sized waterless vaporizing devices.

It is still another object of the present invention to provide a kit for a vaporizing device that has a bottle with one or more scented oils that allows the user to selectively choose a desired scent for selective application to the pad and to selectively choose the fit of the pad to adequately fit in a number of differently sized waterless vaporizing devices.

It is still yet another object of the present invention to provide a kit for a vaporizing device that could have a number of different oils and number of sized pads.

It is a further object of the present invention to provide a kit for a waterless vaporizing device that has a number of different scented oils with or without any water therein.

It is a further object of the present invention to provide a kit to enable consumers to make “refills” for all waterless vaporizers.

It is a further object of the present invention to provide a kit to allow the combination of any type or size of pad with any type of liquid in one kit.

It is a further object of the present invention to provide a kit to allow the user to make their own refill pad to fit into any type or brand of waterless vaporizer.

It is a further object of the present invention to provide a kit to enable the consumer to “refill” the pad that came with their particular waterless vaporizer.

It is a further object of the present invention to provide a kit that has one or more bottles or containers of oil and one or more sizes of pad.

These and other objects and advantages of the present invention are achieved by a kit of the present invention. The kit has a receptacle or receptacles for holding an amount of scented material. The kit also has one or more sized pads. The sized pads are applied with one or more scented materials and may be selectively sized to universally fit in any number of differently sized waterless vaporizer devices.

### DESCRIPTION OF THE FIGURES

FIG. 1 is a perspective view of a preferred embodiment of a waterless vaporizing device.

FIG. 1A is a cross sectional view of the waterless vaporizing device showing a compartment for a refill pad to be placed in or on.

FIG. 2 is a top perspective view of a kit with a number of receptacles having an amount of scented material being disposed therein.

FIG. 3 is a top perspective view of the kit having one or more pads for selectively installation into the waterless vaporizer of FIG. 1.

FIG. 4 is a view of the kit of the present invention.

FIG. 5 is a view of a carton for transport of the kit.

### DETAILED DESCRIPTION OF THE PREFERRED EMBODIMENT

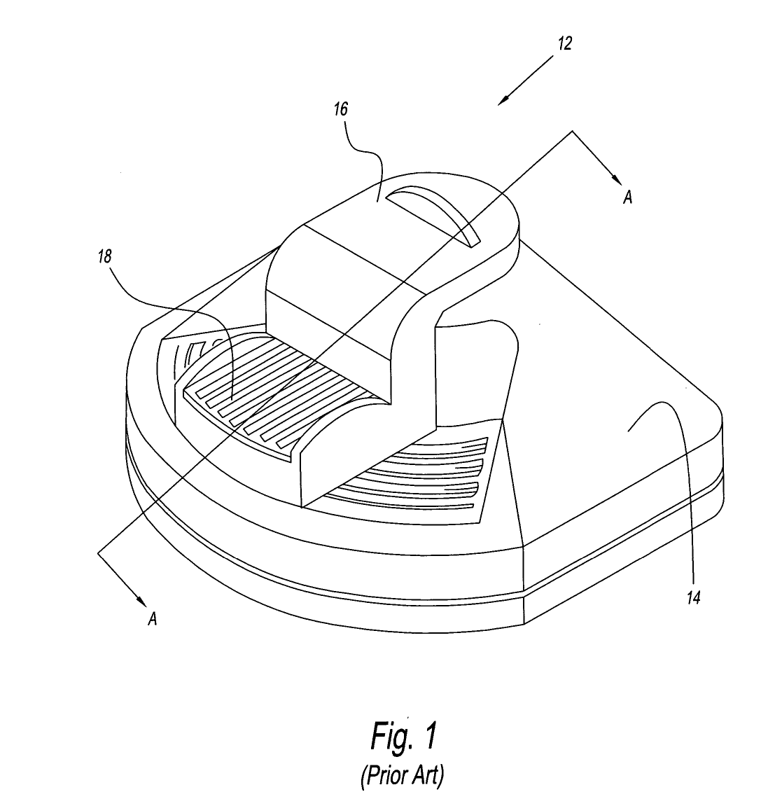

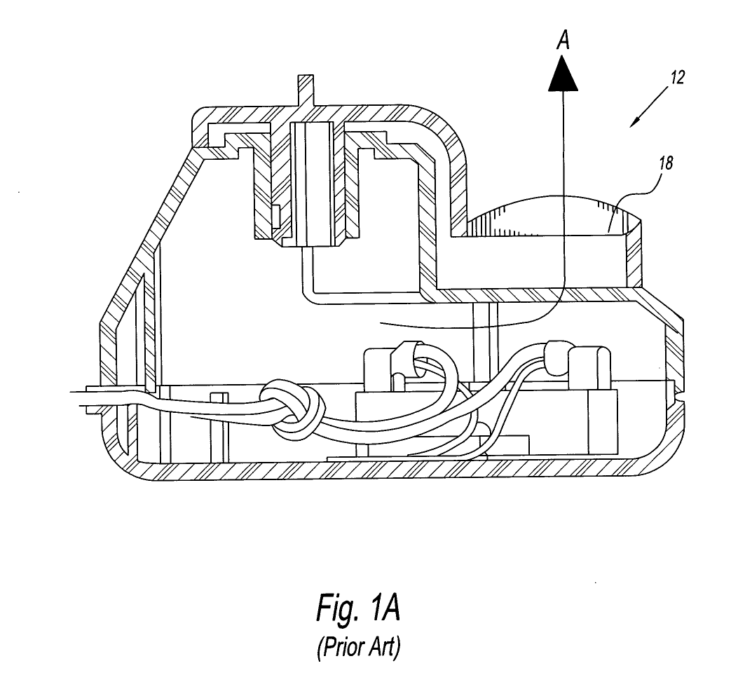

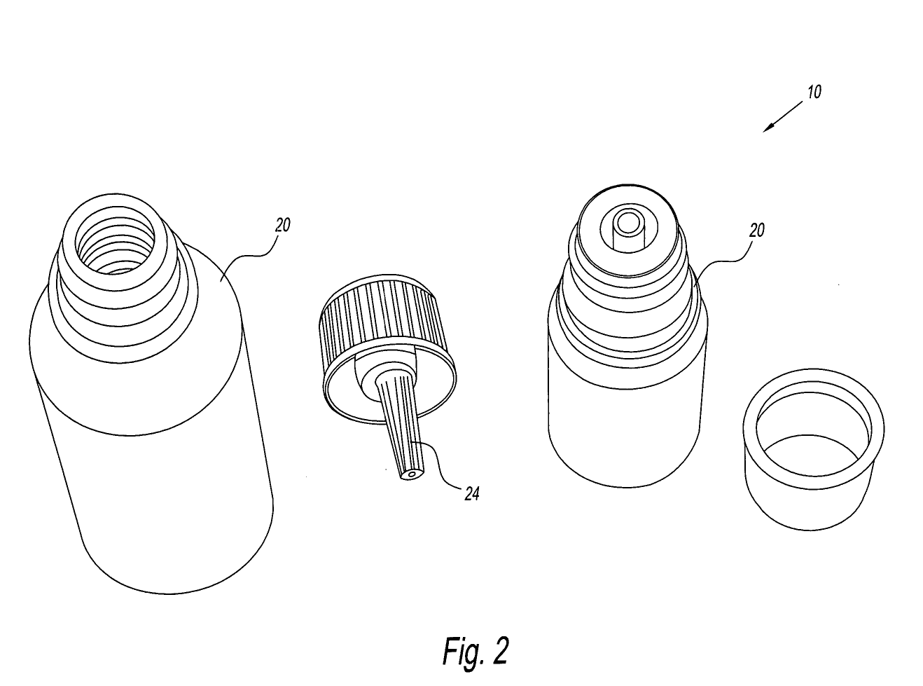

Referring to the figures and in particular FIG. 2 through 5, there is shown a kit of the present invention generally represented by reference numeral 10. Referring to FIG. 1, there is shown a waterless vaporizing apparatus generally represented by reference numeral 12. The waterless vaporizing apparatus 12 is shown as an example only and the present invention is in no way limited to the embodiment shown. In fact, the kit 10 of the present invention is intended to be universally used with multiple different types and different sized waterless vaporizing devices such as a mini waterless vaporizer, a plug in device, an appliance intended to be used or function as a waterless vaporizer, or any other device known in the art and any combinations thereof for producing a thermally induced convection air flow through a pad for producing a scented vapor.

Referring to FIG. 1, there is shown the waterless vaporizing apparatus 12 having a housing 14. The housing 14 has a heater (not shown) disposed in the housing and a chimney 16 having one or more vents 18. The apparatus 12 preferably produces a thermally induced convection air flow through a pad for producing a scented vapor through the one or more vents 18. Referring to FIG. 1A there is shown a cross sectional view of the apparatus 12 along line A-A. The chimney 16 may be opened or closed and has a lateral space or compartment 18 thereon for inserting a pad therein. The pad is impregnated with a scented oil. The waterless vaporizer 12 further has the air flow being disposed through the waterless vaporizer and further through the pad shown by reference arrow A. Thus, a predetermined scented air flow or vapor A can be emitted from the waterless vaporizing apparatus 12.

One significant problem in the art is that the compartment 18 for storing the pad or refill pad is usually sized so as to only allow a specific or predetermined sized pad to fit therein. Thus, user will have difficultly in any attempt to change the pad or change the scents that is emitted from the pad that fits in the compartment 18 and will be forced to purchase predetermined sized pads. Moreover, the user is forced to use only scents offered by the manufacturer of the apparatus 12.

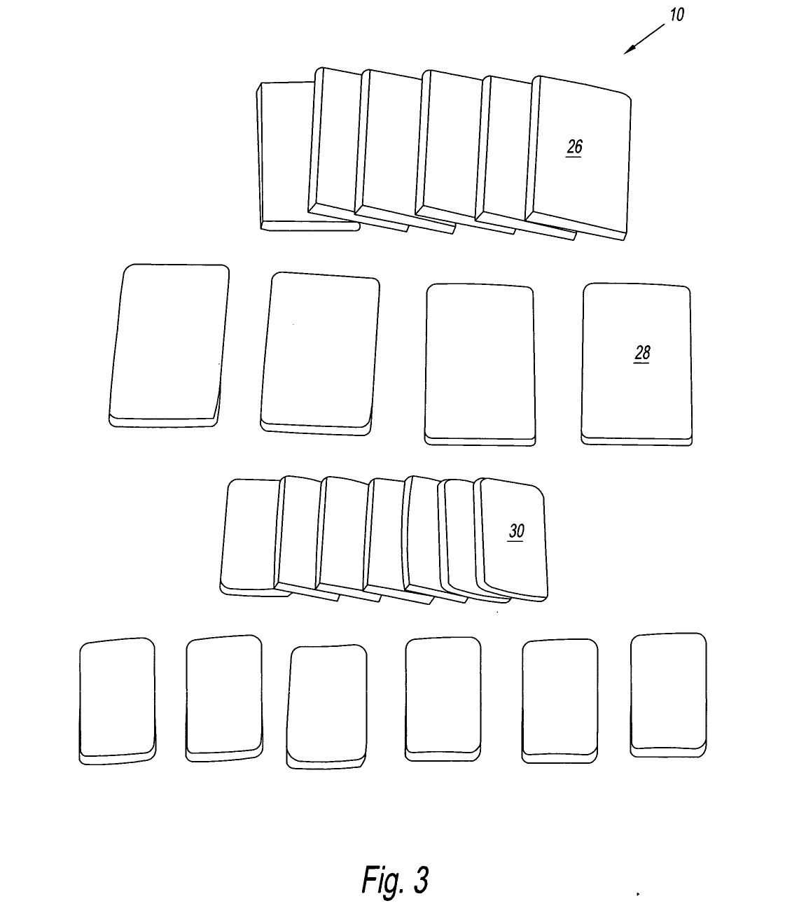

The kit 10 of the present invention remedies this problem in the art in an unexpected manner. Typically, water based vaporizers have the ability to change scents merely by the user pouring a scented liquid in the water in a reservoir of the water based vaporizer. The waterless vaporizers do not have any such configuration and are precluded from doing so. However, the kit 10 of the present invention, preferably allows the user to modulate the scent emitted from the waterless vaporizing apparatus 12. The kit 10 further allows the user to match the size of the refill pad and/or universally size the desired refill pad to any apparatus 12 to give the user a great amount of flexibility in using the apparatus. Referring to FIG. 2, the kit 10 has a number of bottles 20 and a number of pads 22 (shown in FIG. 3) stored in a carton 24. The number of bottles 20 are shown in FIG. 2 as two bottles, however, the kit 10 may have any arrangement of scented material in any number of receptacles or bottles. For example, the bottles 20 may hold an oil, a scented water, a perfume, eucalyptus oil, a menthol, a lavender scent, camphor, an aromatic crystalline compound, an organic compound, an inorganic compound, another aroma bearing material known in the art or any mixtures thereof.

The kit 10 has one bottle 20 or several bottles of liquid containing the scented material. The scented material preferably is an oil, a homeopathic ingredient, a homeopathic ingredient with an active or inactive ingredient, a homeopathic ingredient having water, or any combinations thereof. This bottle 20 can be provided in any size or shape however it is preferably twenty five milliliters for a predetermined number of uses. Preferably twenty five milliliters of scented material allowing about 1.7 milliliters of scented material per use will allow the user to apply an amount on at least fifteen pads however the kit 10 is not limited to this arrangement. In one preferred embodiment, the kit 10 holds fifteen blanks pads 22. Preferably, the scented material has an amount of oil and not water to ensure that each pad 22 of the kit 10 gets more “fills” and has a pleasing scent.

The bottle 20 preferably is made from a resilient material and is most preferably made from either a plastic material or glass. The bottle 20 also preferably has a tamper proof seal, and an applicator 24. The applicator 24 is preferably a dropper, or dripper, however it also may have a separate discrete dropper therewith not connected to any cap. The kit 10 further has a compressible seal under a cap for the bottle to provide for long shelf life and to prevent spills and deterioration of the scented material therein.

Referring to FIG. 3, the kit 10 further has a number of pads 22 with the number of pads having a preselected size or possibly a first size 26, a second size 28 and a third size 30 in one preferred embodiment. Most preferably, the kit 10 has pads in a of size to universally fit existing vaporizer apparatuses, mini-waterless vaporizers, plug in devices, and any appliance intended to function as a waterless vaporizer or that forms a thermally induced convention air flow through the pad 22. The pads 22 most preferably, are made in a range of sizes. In one embodiment of the present invention, the pads 22 have a configuration of ¾ inch by 1.5 inch with about a 3/16 inch thickness, and allows for the trimming of the pads (22) to fit any of the above conveyances, but once again, the present invention has the kit 10 with pads suitable for use in any type of waterless vaporizer and also contains any type of liquid suitable for filling the pad. Alternatively, the kit 10 further may optionally have a cutting tool and/or instructions for being able to size each of the pads 22. In this manner, the user can size the pad 22 for universal installation with any waterless vaporizer apparatus 12 known in the art. The kit further has the pads 22 being made from one hundred percent cotton. Alternatively, the kit 10 may have the pads 22 made from any other absorbent material known in the art intended to hold the scented material such as a nylon, a rayon, or any other absorbent material or cloth known in the art.

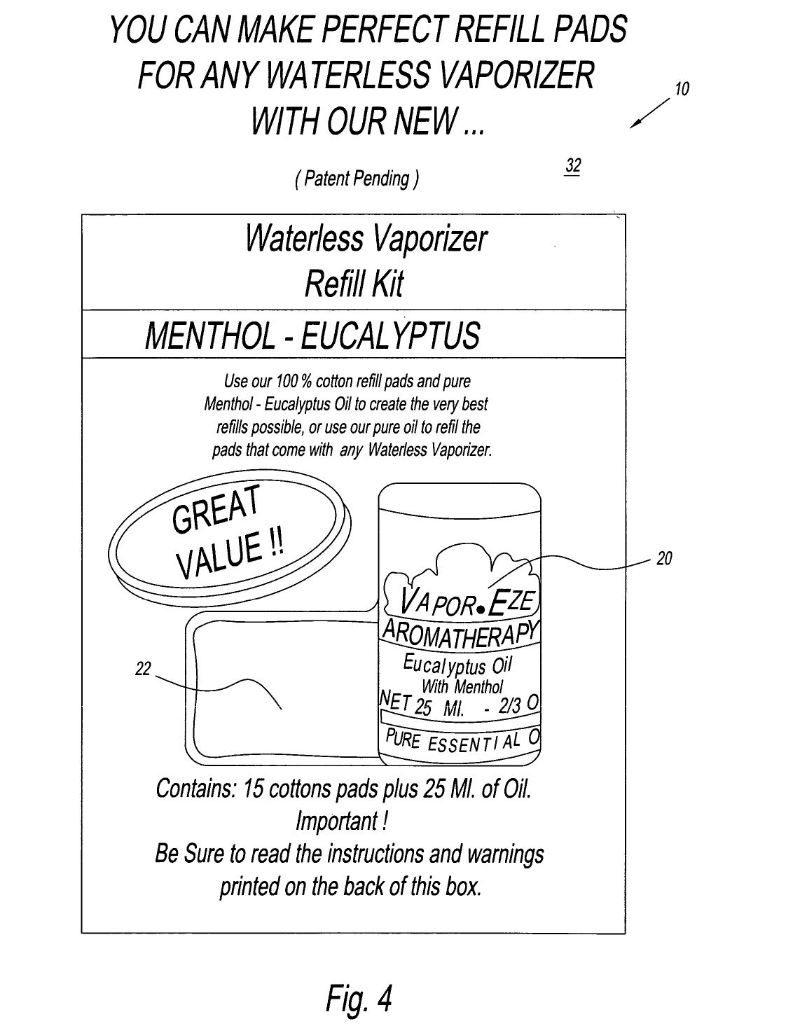

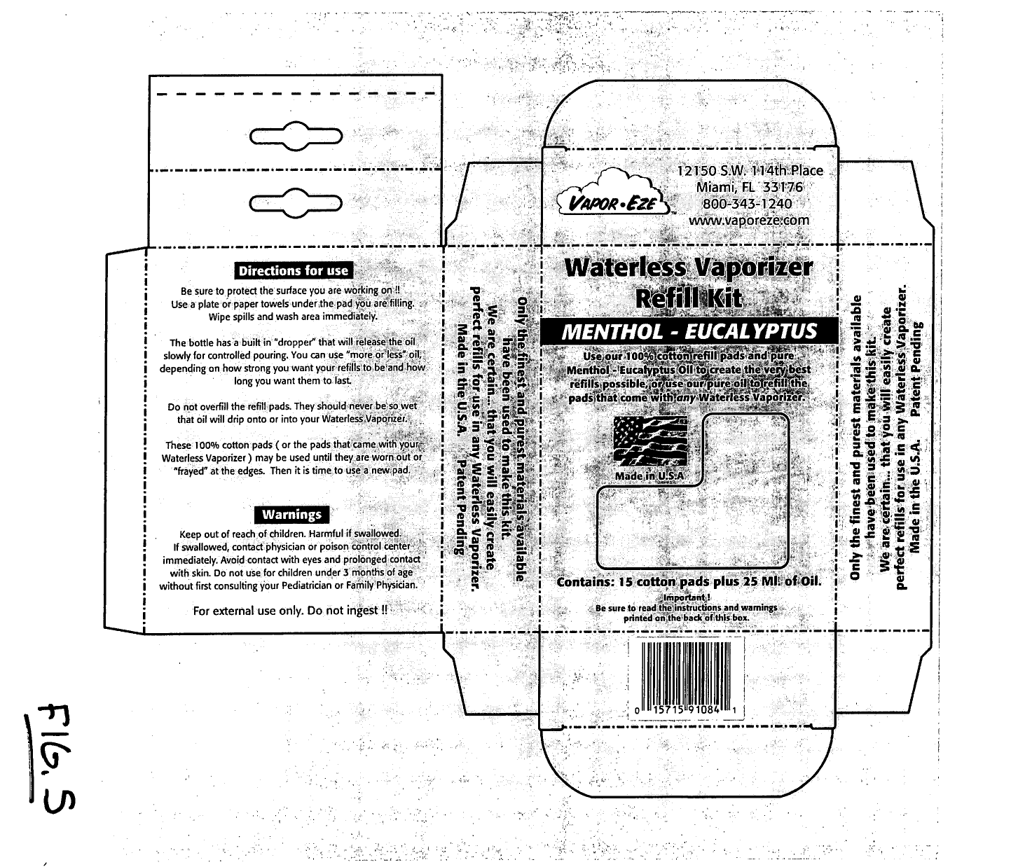

Referring now to FIGS. 4 and 5, there is shown an exemplary carton 32 being used with the kit 10 of the present invention. The carton 32 is preferably sealed and has a number of pads 22 and a number of bottles 20 as discussed herein. It is within the scope of the present invention to provide a transportable kit 10 having any number of bottles 20 or pads 22 for a universal sizing of substantially all refill pads sold for existing waterless vaporizers 12.

It should be understood that the foregoing description is only illustrative of the present invention. Various alternatives and modifications can be devised by those skilled in the art without departing from the invention. Accordingly, the present invention is intended to embrace all such alternatives, modifications and variances.

What is claimed is:

1. A kit for replacing a refill pad for a waterless vaporizing unit, the kit comprising:
a pad; and
a scented material for being deposited on said pad, wherein said pad is for a universal replacement for a number of different sized waterless vaporizing units.

2. The kit of claim 1, wherein said pad is selectively sized to fit in the vaporizing unit.

3. The kit of claim 1, wherein said pad is an absorbent material.

4. The kit of claim 1, wherein said scented material is a liquid .

5. The kit of claim 4, wherein said scented liquid comprises menthol.

6. The kit of claim 4, wherein said scented liquid comprises eucalyptus oil.

7. A kit for a universal replacement of a refill pad for a vaporizing unit, the vaporizing unit having a vaporizing unit sized compartment for the refill pad, the kit comprising:
a pad; and
a liquid for being deposited and saturating said pad, and wherein said pad is selectively sizable to universally fit a plurality of differently sized waterless vaporizing unit compartments.

8. The kit of claim 7, wherein said pad has an initial size, said initial size being reduced to fit in each of said plurality of differently sized waterless vaporizing unit compartments.

9. The kit of claim 8, wherein said liquid is a scented liquid .

10. The kit of claim 9, wherein said liquid comprises menthol..

11. The kit of claim 1, wherein said liquid comprises eucalyptus oil.

12. A method of replacing a refill pad for a waterless vaporizing unit, the method comprising:
providing a pad and a receptacle having a scented material in a box;
removing and discarding a used pad;
sizing the pad for the vaporizing unit having a predetermined size;
depositing on said pad the scented material, and replacing the used pad with the sized pad.

13. The method of claim 12, wherein the step of sizing the applicator is provided by a cutting tool.

14. The method of claim 13, wherein the step of depositing the scented material is provided by a dropper.

15. The method of claim 12, wherein said pad is cotton.

16. The method of claim 12, wherein said scented oil is selected from the group consisting of a scented oil, menthol, eucalyptus oil, and any combinations thereof.

17. The method of claim 12, wherein the step of sizing the pad is provided by a manner selected from the group consisting of selecting from a plurality of differently sized pads, trimming said pad, and any combinations thereof.

18. The kit of claim 1, further comprising a cutting tool for sizing said pad based on said plurality of vaporizing units with each of the plurality of vaporizing units being a differently sized unit .

19. The method of claim 14, wherein said dropper is integral with a receptacle for holding said scented.

20. The kit of claim 1, further comprising a plurality of pads with each of said plurality of pads having a size, wherein some of said plurality of pads have a size being different for fitting in said plurality of different waterless vaporizing devices.
